(19) United States
(12) Patent Application Publication
(10) Pub. No.: US 2024/0230544 A1
(43) Pub. Date: Jul. 11, 2024

(54) DEVICES, SYSTEMS, AND METHODS FOR MEASURING ELECTROLYTE CONCENTRATION IN BIOLOGICAL FLUIDS

(71) Applicant: Jana Care, Inc., Boston, MA (US)

(72) Inventors: Daniel B. Lookadoo, Boston, MA (US); Ashok A. Kumar, Malden, MA (US); Jeremy E. Schonhorn, Hopkinton, MA (US); Harshit Harpaldas, Farmington, CT (US); Chrisptoher M. Uherek, Boston, MA (US); Michal Depa, Beaconfield (CA)

(21) Appl. No.: 18/563,835

(22) PCT Filed: May 31, 2022
(86) PCT No.: PCT/US2022/031603

§ 371 Date: Nov. 22, 2023

Related U.S. Application Data
(60) Provisional application No. 63/194,546, filed on May 28, 2021.

(51) Int. Cl. G01N 21/78; G01N 21/77; G01N 33/52; G01N 33/84

(52) U.S. Cl. CPC G01N 21/78; G01N 33/525; G01N 33/84; G01N 2021/7759; G01N 2201/0221

(57) ABSTRACT

Systems, methods, and measuring devices by which an analyte, and more specifically electrolytes such as, for example, potassium, can be selectively extracted and measured easily and in a minimally invasive manner using a small amount of biological fluid, such as blood, plasma, or serum. The measuring device or chemical sensor is a test strip including a layered active component assembly sandwiched between two optional film or support layers. The layered active component assembly includes ion-sensitive membranes for performing ion-selective extraction methods. The test strips can be utilized with an optical reader connected to a smartphone or other mobile device for remote monitoring of an analyte level associated with conditions of a disease.

### RELATED APPLICATION

This application claims the benefit of U.S. Provisional Application No. 63/194,546 filed May 28, 2021, which is hereby fully incorporated by reference herein.

### BACKGROUND

Chronic conditions, such as chronic kidney disease and heart failure, give rise to increased burdens on health care systems resulting in increased health expenditures and preventable deaths. These conditions require essential electrolyte monitoring for management of these conditions. For example, electrolyte concentration, such as the potassium ion content of a subject's blood, can provide critical clinical indication of many of these conditions. Specific to potassium, potassium cations are one of the most abundant elements found in intracellular and extracellular media. Excessive potassium levels, known as hyperkalemia, and deficient potassium levels, known as hypokalemia, are both responsible for various symptoms, including severe cardiac electrophysiology alterations such as potentially fatal cardiac arrhythmias. Various methods for quantitatively determining the blood electrolytes have been developed, including adsorption/emission spectroscopy, capillary electrophoresis, and ion-selective electrodes (ISEs). These methods are typically implemented with special instruments which can be difficult to operate and often require additional calibration.

Ionophore-based ion selective optical sensors, or ion selective optodes (ISOs), use two separate entities to recognize and report the presence of the target ion, or broadly, target analyte. An ionophore is used for ion recognition, and a chromoionophore (typically a lipophilic pH indicator) produces the optical reporter on the basis of ion exchange. More specifically, as the target ion level in the sample increases, the hydrogen ion concentration in the sensor decreases via an ion-exchanger, such that the electrolyte activity can be detected indirectly by monitoring the hydrogen ion level. Ion selective optodes are described, for example, in Xie et al., “Ion selective optodes: from the bulk to nanoscale,” Anal. Bioanal. Chem., January 2015, incorporated herein by reference in its entirety.

Optode potassium tests have been developed to measure potassium levels in a blood sample. Recent research suggests that optode tests are particularly adaptable for blood analysis. Such tests include ion selective nanosensors that work in either equilibrium or exhaustive modes. In equilibrium mode, the analyte concentration is not altered. In exhaustive mode, analyte ions are completely consumed by the sensors.

European Patent No. 0207392 to Charlton et al. and European Patent No. 0229982 to Genshaw teach the use of a secondary, inert colorant for reducing the variability in test results. These patents disclose methods for reducing both device to device variability and strip to strip variability through use of an inert secondary colorant. However, neither of these patents contemplates a system that operates in an exhaustive mode where the ionophore is sufficiently in excess such that all target ions are bound.

Another solution provided in the prior art can be exemplified by a reflectron system developed by Roche. The Roche reflectron is described, for example, in U.S. Pat. No. 5,211,914 to Vogel. The Roche reflectron measures ion concentration using valinomycin as an ionophore. The Roche device uses a test layer comprising a liquid resistant film that is coated on a uniform layer onto a clear film. Roche teaches that clear film is critical, to ensure precision of the optode test results. In practice, however, the film layers described by Roche are challenging to manufacture and the chemical mechanism is extremely slow.

U.S. Patent Application 2014/0162372 to Park et al. discloses a cartridge-based system with an optode substrate in contact with a buffer substrate to control for pH of the optode. One major disadvantage of sensors such as those disclosed by Park is that the response is known to be pH dependent. The sample pH, therefore, needs to either be kept constant or monitored at the same time as the ion concentration. Such pH cross response has significantly limited the application of this class of sensors.

Exhaustive mode optodes have also been disclosed in the prior art. The response from a sensor operating in exhaustive mode should depend only on the total amount of analyte present in the sample, making the exhaustive sensors potentially calibration-free. Unfortunately, the exhaustive mode optode sensors contemplated by prior art publications have struggled to demonstrate a simple method of embedding all required elements in a paper test strip construct. Some examples describe systems in which the chemical mechanism is performed in a series of vials. Other publications disclose performing some of the reaction in wet chemistry, then adding the solution to the substrate to perform the test on paper. These descriptions demonstrate the difficulty and challenges of developing an exhaustive sensor that can operate in a dry chemistry design, using a paper-type sensing layer.

Thus, there is a need in the art for a dry chemistry design of an optode sensor operating in exhaustive mode and designed for use with a paper test strip for point-of-care (POC) and self-testing and monitoring of a condition. Such a device would improve the efficacy of optode potassium test systems and methods.

### SUMMARY

Embodiments are directed to systems, methods, and measuring devices by which an analyte, and more specifically electrolytes such as, for example, potassium, can be selectively extracted and measured easily and in a minimally invasive manner using a small amount of biological fluid, such as blood, plasma, or serum. The measuring device or chemical sensor, according to embodiments, comprises a test strip including a layered active component assembly sandwiched between two optional film or support layers. The layered active component assembly includes ion-sensitive membranes for performing ion-selective extraction methods. The test strips of the embodiments can be utilized with an optical reader connected to a smartphone or other mobile device for remote monitoring of an analyte level associated with conditions of a disease.

In embodiments, the test strip incorporates a vertical flow assay that includes an ion-selective optode (ISO) to produce a colorimetric signal proportional to the concentration of a target ion in the sample. ISOs are photometric counterparts to ion-selective electrodes (ISEs): they rely on the same ion-selective extraction chemistry but respond to a pH indicator dye instead of an indicator electrode. The test strip can be used with an optical sensing device or optoelectronic reader that is a compact, handheld device couplable to or containing an analyzer device (or reader device) such as a mobile phone or tablet device and is capable of using diffuse reflectance to measure a color change in a test strip for quickly detecting and measuring electrolyte concentration, e.g., potassium concentration. The analyzer device includes algorithms to quantify the electrolyte concentration and to analyze signals to compensate for sample and test variability, to detect errors in test procedure, and optionally, to detect sample lysis.

In certain embodiments, the test strip generally includes a top film layer having structure defining an aperture for application of the sample, optional foam spacers placed on both sides of the application pad to provide support and structure across the test strip, an assembly of active or functional material layers assembled beneath the top layer, and a bottom film layer coupled to the top layer to sandwich the functional material layers therebetween. The functional material layers include an optional sample application pad or spreading mesh coupled to an inner-facing surface of the top film layer below the aperture, a plasma separation membrane coupled to the spreading mesh, and an optode membrane substrate coupled to the plasma separation membrane. The optode membrane substrate includes a formulation of an ionophore, an ion-exchanger, and a chromoionophore embedded within a lipophilic environment to enable phase separation and ion-selective extraction from the sample. The optode ion exchange, described above, occurs on the optode membrane such that a colorimetric signal proportional to the electrolyte concentration or target ion is produced. The top and bottom film layers are coupled together to sandwich the active layers therebetween and to provide a barrier to prevent liquid from leaking out of the test strip during use.

The layers can be bonded together by any of a variety of bonding techniques, such as, for example, adhesives, heat sealable materials, or ultrasonic welding. In a particular embodiment, an optically clear adhesive layer is present between the detection layer and the bottom film layer so as not to interfere with optical signal detection. In an embodiment, the first and second film layers define the two outermost layers of the composite test strip, however, in alternative embodiments, additional layers and/or coatings can be incorporated as desired.

A kit and a method for using the kit for measuring an electrolyte concentration in a biological fluid to monitor a condition, according to embodiments of the invention, includes a plurality of test strips, and a set of instructions for preparing the test strip for measurement using an optical sensing device coupled to or incorporated into an analyzer device.

According to embodiments, a method for measuring an electrolyte concentration can include obtaining a blood sample, applying the blood sample to the application pad of the test strip, and inserting the reacted test strip into an optical sensing device coupled to or incorporated into an analyzer device for measurement and analysis. The method can further include installing an application on a mobile device, pairing the mobile device with the optical sensing device, and collecting, reading, and/or analyzing the data in the application on the mobile device.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is an exploded perspective view of a test strip assembly according to an embodiment of the invention.

FIG. 2A is an exploded cross-sectional view of the test strip assembly of FIG. 1.

FIG. 2B is a cross-sectional view of the test strip assembly of FIG. 1.

FIG. 3 is an example of spectra generated from test strips and methods according to an embodiment of the invention at different concentrations of potassium in a sample.

FIG. 4 is a graph demonstrating the linear response of a potassium optode according to an embodiment with a buffer sample.

FIG. 5 is absorbance spectra of K+-selective optodes prepared with different chromoionophores.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

The embodiments described below are not intended to be exhaustive or to limit the invention to the precise forms disclosed in the following detailed description. Rather the embodiments are chosen and described so that others skilled in the art may appreciate and understand the entire disclosure.

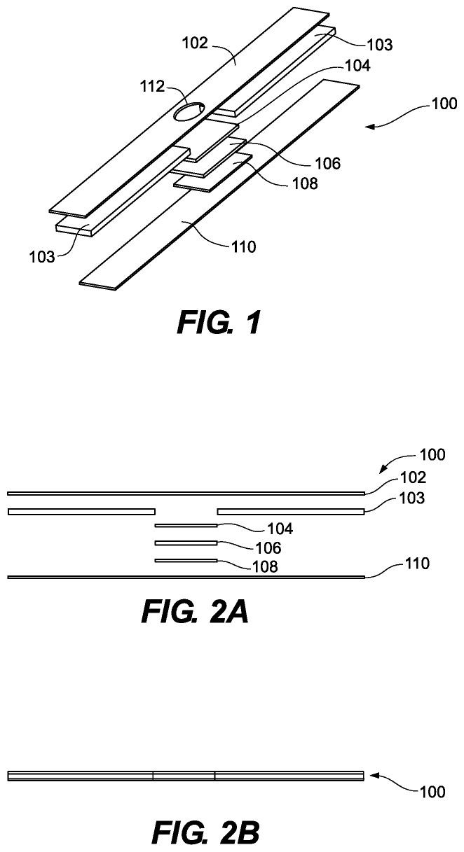

Referring to FIGS. 1, 2A, and 2B, an apparatus for detecting an electrolyte concentration in a biological fluid, such as blood, serum, or plasma, comprises a composite test strip assembly 100 used for applying a sample and for inserting such sample laden strip into an optical sensing and reading apparatus for analysis of the sample. In the embodiment depicted in FIGS. 1, 2A, and 2B, test strip assembly 100 comprises six layers. In alternative embodiments, more or less than six layers can be contemplated.

Test strip assembly 100 can comprise an optional first or top hydrophobic film layer 102 having structure defining an aperture 112, a spreading mesh application pad 104 coupled to top film layer 102 below aperture 112, one or more optional inert, hydrophobic foam spacers 103 coupled to the top film layer on each side of application pad 104 to provide support and structure to the test strip assembly 100, a plasma separation membrane 106 such as a filter paper (e.g., high purity cotton linter paper such as CytoSep HV) positioned below application pad 104, a detection layer 108, such as a treated membrane or substrate, positioned below separation membrane 106 and configured to induce a colorimetric response to the presence of the target ion, such as potassium, and a second or bottom film layer 110 operably coupled to top film layer 102 to sandwich components 104, 106, and 108 therebetween and to provide optical access to the colorimetric response that occurs upon sample introduction to detection layer 108.

Optional top film layer 102 or backing card can be formed from a plastic or polymeric material that exhibits a balance between a moderate flexural modulus (e.g., from about 100,000 to about 600,000 psi), and good tensile strength (e.g., from about 3000 to about 15000 psi). This allows for ease in manufacturing, while yet providing a composition test strip assembly 100 rigid enough to withstand the operational handling involved in performing assays. Top film layer 102 is also hydrophobic and non-porous so as to not interfere with the fluid sample moving through the test strip assembly. Suitable materials include, for example, polyethylene terephthalate (PET), vinyl materials, acetal copolymer, acrylic, nylon, polyester, polypropylene, polyphenylene sulfide, polyetheretherketone, poly(vinyl chloride), or combinations thereof.

Optional foam spacers 103 are formed from inert, non-wicking, hydrophobic foam material that provides spacing for the active layers 106, 108, and acts as a buffer to provide uniform pressure across the assembly 100. Spacers 103 allow for control of fluid transfer between and through the layers without the need of an external cassette (cartridge) or housing.

Application pad 104, in the form of a spreading mesh, provides capillary force for directing flow of the fluid sample to plasma membrane 106. The material of application pad 104 is selected to reinforce membrane 106 and to provide uniform wetting across separation membrane 106, and can include, for example, a one-direction or multi-direction mesh or woven materials with consistent structure, thickness, and porosity. The material used in application pad 104 can optionally be treated with a hydrophilic treatment to provide sufficient spreading of the fluid sample. The material of application pad 104 can comprise, for example, nylon, fiberglass, a superabsorbent polymer such as a hydrogel, cellulose, or combinations thereof. In one embodiment, a monofilament yarn made of polyester or polyamide is selected. In some embodiments, the material selected for use in application pad 104 has a large percent open area to minimize dead volume, such as an open area percent in a range of from about 13% to about 71%, and more particular from about 43% to about 52%. One commercially available material suitable for use in application pad 104 is SAATICARE Hyphyl Polyester (105/52).

Separation membrane 106 can comprise a plasma separation membrane material or filter paper and is chosen to be compatible with the selected electrolyte detection mechanism, while minimizing lysis (i.e., rupturing of the blood cells), and dwell volume. The material is also selected to avoid dehydration of cells (cell shrinkage) and/or otherwise to avoid causing the release of intracellular electrolytes into the filtered serum to be tested. Suitable materials for use in plasma separation member 106 are commercially available as Pall Vivid Plasma Separation Membrane, Ahlstrom Cytosep HV, and Ahlstrom 169.

Detection layer 108 comprises a treated substrate comprising paper, a membrane, plastic sheet, and/or mesh, chosen for uniformity of color production. The substrate is coated or otherwise treated with an optode coating solution and additional excipients to aide in the re-suspension of the optode coating solution upon contact with the wet fluid sample moving through the strip assembly. The optode coating solution is formulated to carry out ion exchange chemistry for electrolyte detection, such as potassium detection, as described above. The optode coating solution, when in the presence of potassium (or other target ion as contemplated), produces a colorimetric signal which is proportional to a concentration of the electrolyte in the sample.

In embodiments, the optode coating solution, such as an emulsion-based solution or surface coated polystyrene microspheres, comprises a chromoionophore cocktail configured to detect an electrolyte in exhaustive mode in which the sample analyte or target ion is ideally completely consumed by the ionophore, assuming that there are more ionophore binding sites in the cocktail than amount of target ion in the sample. In embodiments, a suitable chromoionophore cocktail can comprise an ionophore selective to the target ion, an ion-exchanger, a lipophilic core material, a chromoionophore selective to a proton, an amphiphilic polymer, and a carrier solvent.

In embodiments, suitable ionophores can comprise valinomycin, nigericin, or bis(benzo-5-crown-5) compounds such as 2-Dodecyl-2-methyl-1,3-propanediyl bis[N-[5′-nitro(benzo-15-crown-5)-4′-yl]carbamate] (BME 44), Bis[(benzo-15-crown-5)-4′-ylmethyl] pimelate (BB15C5), either alone or combinations thereof. In embodiments, suitable ion-exchangers can comprise organic analogs based on tetraphenylborate (e.g., tetrakis[3,5bis(trifluoromethyl)phenyl]borate, TFPB) or tetraalkylammonium salts (e.g. tridodecylmethylammonium, TDMA), either alone or combinations thereof. In embodiments, suitable lipophilic core materials can comprise bis(2-ethylhexyl)sebacate (dioctylsebacate, DOS), dibutyl sebacate (DBS), dodecyl 2-nitrophenyl ether (o-NPDDE), o-nitrophenyloctylether (o-NPOE), either alone or combinations thereof. In embodiments, suitable chromoionophore materials can comprise chromoionophore I/ETH 5294 (9-(Diethylamino)-5-(octadecanoylimino)-5H-benzo[a]phenoxazine, N-Octadecanoyl-Nile blue, 3-Octadecanoylimino-7-(diethylamino)-1,2-benzophenoxazine), chromoionophore II/ETH 2439 (9-Dimethylamino-5-[4-(16-butyl-2,14-dioxo-3,15-dioxaeicosyl)phenylimino]benzo[a]phenoxazine), chromoionophore III/ETH 5350 (9-(Diethylamino)-5-[(2-octyldecyl)imino]benzo[a]phenoxazine), or oxazinoindoline (Ox) dyes (Ox), either alone or combinations thereof.

A non-limiting example of a chromoionophore cocktail for detection of potassium comprises: a macrocyclic ionophore possessing neutral oxygen donors, such as dibenzo-18-crown-6 or cryptand-222, with valinomycin being the preferred ligand; a negatively charged ion-exchanger, such as, for example, sodium tetrakis [3,5-bis(trifluoromethyl)phenyl]borate; chromoionophore I, a lipophilic core composed of dioctyl sebacate alone, or of polystyrene, acrylic or plasticizer such as dioctyl sebacate with a high molecular weight poly(vinyl chloride) or a polyether compound such as polyethylene glycol, pluronic F-127 (nonionic, surfactant polyol), or Brij-35 9 (non-ionic and zwitterionic detergent for protein solubilization); and a carrier solvent capable of solubilizing the cocktail components and being miscible with water, the carrier solvent including materials such as DMF, acetone, or THF.

As mentioned above, in embodiments, the chromoionophore cocktail further includes suitable excipients that are configured such that when the cocktail is dispensed and dried on a substrate, it can be quickly and uniformly rehydrated or re-suspended when contacted with the wet sample (etc. buffer, serum, plasma) moving through the test strip assembly. Suitable excipients can include, for example, sucrose, glycerol, or trehalose.

In one embodiment, the chromoionophore cocktail is directly dispensed onto the substrate, such as by coating, spraying, additive printing, or any of a variety of dispensing methodologies. In another embodiment, the chromoionophore cocktail is dispensed on beads, such as polymer beads, by coating, spraying, or other means, and the beads are dispensed onto or into the substrate.

In embodiments, a buffer is also embedded in the detection layer 108. A suitable buffer is incorporated to normalize variations in inter-individual blood pH and ionic strength. In further embodiments, it is contemplated that additional reagents can be incorporated into detection layer 108 configured to quantify or detect serum hemoglobin as a proxy for sample lysis.

Bottom film layer 110 is optically clear so that the colorimetric response in the reaction zone on detection layer 108 can be detected by a reader. Bottom film layer 110 can comprise, for example, a material the same as or different than the material utilized for top layer 102. For example, bottom film layer 110 can comprise polyethylene, polyvinyl chloride (PVC), polypropylene, polyethylene terephthalate (PET), polytetrafluoroethylene (PTFE), or combinations thereof. Recommend normalizing this list of materials with that provided for the top layer at the first para of page 9. In particular embodiments, the thickness of bottom film layer 110 is in a range of from about 0.1 to about 10 mm. Any adhesive layer(s) (not shown) included to bond bottom layer 110 to optode membrane substrate 108 should also be optically clear. In an embodiment, bottom film layer 110 can comprise a film or paper backing card which includes structure defining a readout window, such that the film or paper itself need not be optically clear. In other embodiments, bottom film layer 110 is eliminated altogether.

In embodiments, test strip assembly 100 is rectangular in shape, and has a length ranging from about 30 mm to about 80 mm. A thickness of top layer 102 can range from about 0.1 mm to about 1 mm, so that when an aperture 112 is present in top layer 102, a reservoir is created for receiving a sample. More particularly, aperture 112 is formed into layer 102 by any of a variety of standard cutting techniques, such as, for example, die cutting or punching, laser cutting, or the like. Aperture 112 can be circular, as depicted, having a diameter ranging from about 2 mm to about 6 mm, such that the reservoir has a sample volume capacity in a range from about 10 μ to about 50 μl. One of ordinary skill in the art would recognize that other aperture geometries can also be contemplated, including, for example, oval, square, rectangular, triangular, etc., with dimensions such that a similar sample volume can be contained. The sidewalls of aperture 112 may be configured to assist with provision of fast and smooth sample flow. For example, the sidewall(s) of aperture 112 can be tapered, concave, convex, or substantially vertical. In some embodiments, aperture 112 is completely bordered by top layer 102, while in other embodiments, at least one side or edge of aperture 112 is not bordered by top layer 102 such that aperture 112 resembles a notch.

In some embodiments, bottom film layer 110 (and/or optionally top film layer 102) comprises printed indicia thereon. The printed indicia can comprise any of a variety of text and/or graphics such as, for example, brand names, logos, instructions, readout messages, warnings, or any combination thereof. In an embodiment, the printed indicia can comprise, for example, text and/or graphics, such as arrows, indicating how test strip assembly 100 is to be inserted into an optical sensing device for measurement.

In the embodiments described above, the top and bottom film layers 102, 110 define the two outermost layers of the test strip assembly. However, in alternative embodiments, more or less layers and/or coatings can be incorporated as desired. For example, in an alternative embodiment, in which there is no aperture formed in the top layer or there is no top layer at all, an optically clear, thin, and rigid bottom film layer as described above defines the bottom layer of the strip assembly. An optode membrane substrate as described above is coupled to the bottom film layer. A plasma separation membrane as described above is coupled to the optode membrane substrate. Finally, an application pad as described above is coupled to the plasma separation membrane. Optionally, adhesive and/or tape on opposing sides of the stack secures the stack together. Optionally, foam spacers as described above can be incorporated into the stack to provide structural support for the design.

In yet another alternative embodiment, the test strip assembly is designed similar to the previous embodiments, wherein the optically clear bottom film layer is replaced by an opaque rigid bottom film layer including an aperture in the area of the reaction zone of the optode membrane substrate allowing optical access to the optode membrane substrate for reading and analyzing.

In embodiments, test strip assembly 100 can be manufactured individually as discrete test strips. Alternatively, a plurality of test strip assemblies can be manufactured in roll form or in a large card format, and upon assembly, individual test strip assemblies are converted or cut therefrom.

In embodiments, a method for analyzing the chemical sensor signal includes algorithms to quantify electrolytes in a biological fluid sample, such as blood, serum, or plasma, and correct for hemolysis. In embodiments, a fluid sample is drawn or otherwise collected from a subject and is applied to the application pad 104 of the test strip assembly 100 via aperture 112 or directly to the application pad 104 if no top film layer is present, according to embodiments of the invention. The test strip assembly 100 is inserted into and read over time and at multiple wavelengths using an optical or color sensing device or reader, which is in turn configured to be coupled to a measuring or analyzing instrument configured to analyze the data produced from the reader, and using algorithms, measure an electrolyte concentration, as described in more detail below.

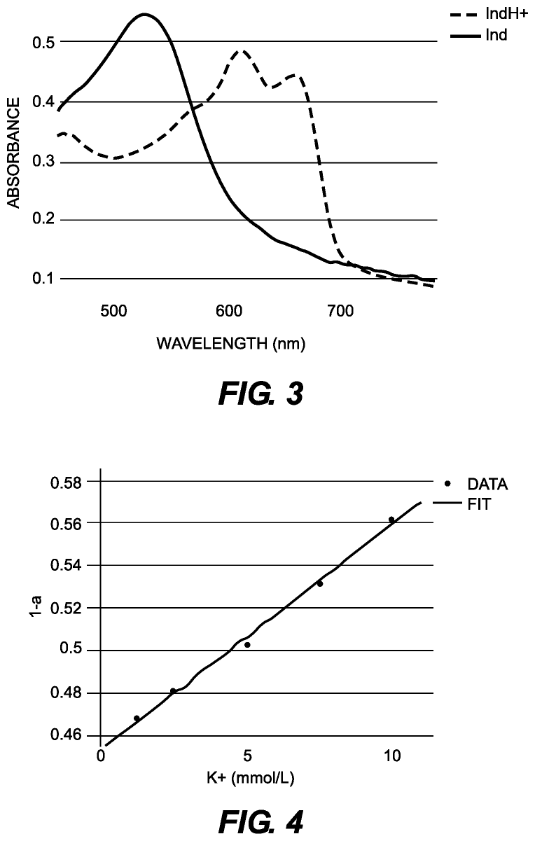

Once data from the reader is collected and analyzed, a spectra may be generated. As depicted in FIG. 3, according to an embodiment, a specific peak depending on the dye chosen can be used to track the concentration of a target ion, such as potassium, in a sample when applied to the test strip. The main peak corresponds to the initial protonated form of the chromoionophore indicator used in the optode. As the target ion binds, charge neutrality dictates that a positive charge (e.g., a proton) must be ejected from the hydrophobic phase, resulting in deprotonation of the chromoionophore proportional to the target ion content as ion exchange occurs. As a result, the absorption peak decreases in the presence of increasing target ion. In the specific case exemplified in FIG. 3, an optional secondary peak corresponding to the deprotonated form of the dye will correspondingly increase with increased target ion content. According to embodiments, two wavelengths are used to correspondingly track the increase and decrease of the two peaks. Using the ratio of the two peaks provides greater sensitivity and also reduces variability between different readers and different test strips.

Referring now to FIG. 4, a graph showing a linear correlation between the optical signal response and the target ion concentration demonstrates the exhaustive mode demonstrated for the first time in a dry chemistry test with no sample preparation steps required according to embodiments of the invention, i.e., the sample chemistry happens on the strip itself as the sample makes its way vertically through the strip. As discussed above, the term “exhaustive mode” refers to nonequilibrium conditions in which the target analyte or ion is completely consumed by the ionophore or sensor locally in the detection zone of the detection layer. This mode of sensing contrasts with conventional platforms that operate under equilibrium conditions.

By measuring the initial and final test strip at multiple wavelengths, variations in manufacturing and activity can be corrected to provide a more precise and accurate reading. Additionally, by measuring the signal over time, the reaction for the electrolyte can be separated out from the reaction for hemoglobin. Further, using a combination of two or more signals (i.e., the spectral response as a function of wavelength for the protonated and deprotonated form of the chromoionophore or indicator) allows for normalization of the signal to be done to compensate for slight variations between different test strips or between different reader devices. In one embodiment, the signals are analyzed using the isosbestic point as a reference point for normalization, while in another embodiment, the first or second derivative of the signals can be used to analyze relative species abundance over background noise in the signal.

Now turning to the sensing device, in an embodiment, the optical sensing device or reader comprises an optical box with a photodiode and two LED light sources chosen to correspond to the chromoionophore, produced in the presence of the target ion, i.e., the ion exchange and deprotonation of the chromoionophore on the detection layer of the test strip assemblies, and to the hemoglobin in the sample. The device, which will be described in more detail below, maximizes diffuse reflectance to capture measurements that are correlated with concentration of the target ion or electrolyte. In an alternative embodiment, the optical sensing device is used with a mini spectrometer in place of the photodiode and a wide spectrum source to provide full spectral imaging of the reaction.

In a particular embodiment, the sensing device can comprise a hand-held reflectance based-optical sensor device, such as a colorimeteric sensor device. Suitable sensing devices can include a device commercially available as the Aina Device, available from the applicant of the present disclosure, and which is described in U.S. Pat. No. 10,436,773 (application Ser. No. 14/997,749) entitled “Mobile Device Based Multi-Analyze Testing Analyzer for Use in Medical Diagnostic Monitoring and Screening,” incorporated herein by reference in its entirety, or the devices and methods depicted and described in U.S. Pat. No. 9,241,663 (application Ser. No. 13/815,764) entitled “Portable Medical Diagnostic Systems and Methods Using a Mobile Device,” also incorporated herein by reference in its entirety. In embodiments, the sensing device connects to any of a variety of mobile devices, such as smart phones or tablets, through the audio jack or jack plug of the mobile device. Although sometimes referred to herein as “jack plug” for sake of convenience, a jack plug can be comprised of any wired or wireless communication element including, but not limited to, universal serial bus (USB), including micro USB and mini USB, Bluetooth®, near field communication (NFC), or WLAN (any IEEE 802.11 variant). The mobile device includes an application that runs on the mobile device for analyzing data generated by the device.

The device may generally include an adapter coupled to an optical sensing body containing optical or color sensing components within (internal, not shown, and as described, for example, in U.S. Pat. No. 10,436,773). The adapter enables the detection layer or detection area of the test strip assembly to align with the optical sensing components housed within the optical sensing body. The adapter includes structure defining a test strip insertion area, such as a slot or channel, for inserting test strips, such as test strip assembly described in the previous sections. When inserted, the test strip assembly is illuminated by one or several light sources, such as two LED light sources, housed within the body. The light reflects from the detection layer of the test strip containing the chromoionophore, which is then measured by a light sensor, such as a photodiode. The reflected color value is then relayed to the mobile analyzing device where it is processed and analyzed by software algorithms contained in the application installed on the mobile device to produce an electrolyte concentration, such as a potassium concentration reading. At each step, appropriate instructions are displayed on the mobile device's screen to guide the user in performing the test.

In an embodiment, the sensing device includes illumination light sources (internal) that allow for bright and consistent illumination, as described in U.S. Pat. No. 10,436,773, incorporated by reference above. One such suitable source of illumination includes through-hole LEDs, which are cost-effective if high luminosity levels are required. To effectively measure the electrolyte concentration on the test strip assembly described previously, the sensing device can comprise at least two separate illumination light sources at different wavelengths.

Optionally, in an embodiment, as the sensing device senses and transmits reflected color data to the mobile device for processing and analysis, the software on the mobile device performs various boundary checking to ensure that the test strip assembly is inserted properly at the different steps, and is not moved during the analysis. These algorithms may include, for example, simple checks such as checking if the reflected value is within a certain expected range, which can be performed simultaneously for the different wavelengths in which the test strip assembly is being analyzed.

### EXAMPLES

Additional data and concepts are provided in the manuscript entitled “Paper-based Optode Devices (PODs) for Selective Quantification of Potassium in Biological Fluids” by Lookadoo et al., incorporated herein by reference in its entirety, and as set forth below.

### Design of Devices

As set forth in the manuscript, test strip assemblies as described herein were designed that had stackable functionality to minimize operator steps, enabled rapid response times to extend exhaustive mode operation, and was simple to fabricate, reducing technology transfer demands for high-volume manufacturing. When a blood sample was applied to the sample ports of the assemblies, it spread uniformly by the mesh across the filter paper, where red blood cell were removed. The spreading mesh of the assemblies had large mesh opening and high open area to allow fast and effective transfer of the sample to the filter paper below. The filter paper (CytoSep HV) had good red blood cell retention and low dwell volume, and was selected for fast blood absorption and plasma separation to mitigate assay interference. A high purity cotton linter paper (Whatman CF1) was selected as the optode carrier for its high rewetting properties to enable exhaustive mode operation. An optically clear film was selected to provide a protective cover for the optode chemistry and hold the stack of materials together. The overall device was designed to minimize blood volume to a reasonably obtainable fingerstick sample (i.e., 15-30 μL).

### Optode Chemistry

The choice of optode chemistry created a strong optical response in the relatively narrow clinically relevant range for K⁺ (about 2.5 to about 6.5 mM) that distinguishes between differences of less than 0.5 mM. The optode chemistry design was chosen that was highly wettable, enabling a uniform colometric signal, had fast response times, and was easily tunable with a linear response to capture the clinically relevant range. As discussed above, the ionophore, ion-exchanger, and chromoionophore were embedded within a lipophilic environment to enable phase separation and ion-selective extraction from the sample. In this example, valinomycin was found particularly suitable as the ionophore for selective complexation of K⁺, which, when selectively extracted from the sample by the ionophore, a proton is released by the chromoionophore to offset the positive charge. The change in protonation degree alters the electronic environment of the chromoionophore, resulting in a measurable color change. The ion-exchanger is used to maintain an electrically neutral environment within the lipophilic core.

The ISOs in this example were formed by a precipitation-based method utilizing surfactants to prepare emulsion-based ISOs rather than surface-coating polystyrene microspheres. This method produces a nano-emulsion that operates in exhaustive mode. Exhaustive mode sensing implies that the electrolyte in the sample is entirely consumed rather than undergoing partition equilibrium.

### Handheld Reader

A portable, handheld and battery powered reader was selected, and more specifically, a smartphone to help minimize reader costs and provide a rich user interface that can detail step by step instructions. Spectral sensing capabilities were enabled to improve the universality of the reader. The core optics included a broad-spectrum white LED and micro-spectrometer assembly that covers the visible spectrum with a resolution of 10 nm or less.

### Chromoionophore Selection

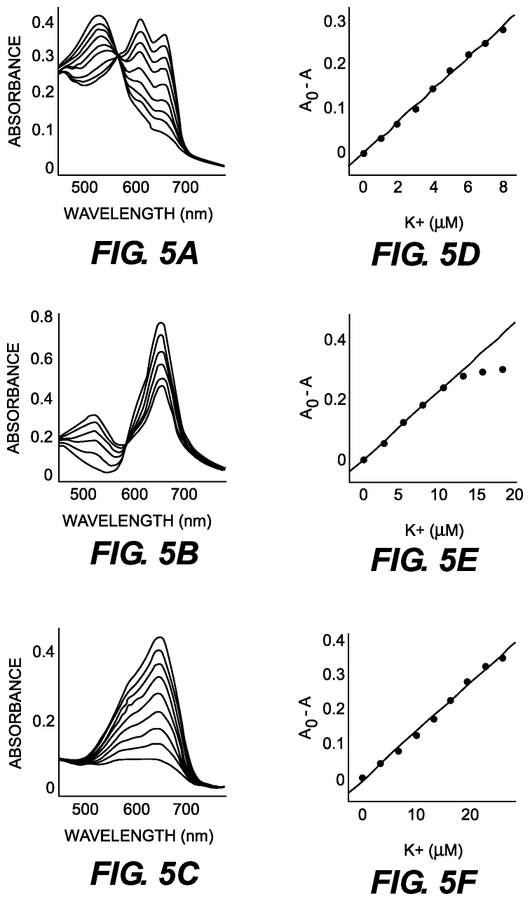

Three chromoionophores were evaluated—CH I, CH III, and Ox B. The three were selected were amendable to exhaustive mode operation, had good signal-to-noise, and maintained functionality when dried down on paper. With the exhaustive exchange ISO system, the change in indicator signal was directly proportional to the total ion concentration. The absorbance spectra of K+ selective optodes prepared with the three chromionophores are depicted in FIG. 5. In the figures, graphs (a)-(c) are CH I, CH III, and Ox B, respectively, whereas (d)-(f) are the calibration curves generated from (a)-(c) by taking the absorbance difference from the buffer blank at 663, 655, and 650 nm for CH I, CH III, and Ox B, respectively. CH I was the preferred chromoionophore due to its ability to account for variable quantities of sensing components and achieve a greater response span than CH III.

Table 1 below provides a statistical summary of the feasibility assessment including expected and measured K⁺ concentration in mM, relative error, and relative standard deviation.

TABLE 1
Statistical Summary of Feasibility Assessment

| Expected | Mean | Rel. Error | rsd |
|---|---|---|---|
| 2.10 | 1.9 | −7.4 | 22.8 |
| 3.10 | 3.3 | +7.4 | 8.1 |
| 4.10 | 4.3 | +5.2 | 6.0 |
| 5.10 | 4.8 | −6.2 | 4.1 |
| 6.10 | 6.4 | +5.1 | 4.5 |
| 7.10 | 6.9 | −2.8 | 3.5 |

Table 2 below compares the different methods for K⁺ analysis using paper-based optode devices, including reader type, test time (sample to answer), exhaustive ranges, and connectivity to a network.

TABLE 2
Comparison of Published Methods for K⁺ Analysis Using Paper-based Optode Devices
Table 1. Comparison of Published Methods for K⁺ Analysis Using Paper-based Optode Devices.

| | | | Test Time | Exhaustive | |
|---|---|---|---|---|---|
| Method | Reader | Process | (minutes) | Range (mM) | Connectivity |
| PODs (this | Handheld | Sample to answer | <2 | 2.0-7.0 | Yes |
| work) | spectrometer | | | | |
| μPADSY⁴¹ | Lightbox and camera | Sample to answer | ~5 | 4.3-6.9 | No |
| Capillary | Scanner | Sample | ~35 | 1.0-6.0 | No |
| Sensors⁴¹ | | aspiration, | | | |
| | | incubation, and | | | |
| | | heating | | | |
| AMES^{58, 59} | Benchtop | Sample dilation, | ~5 | 1.8-10 | No |
| | spectrophotometer | incubation, and | | | |
| | | washing | | | |
| Reflotron⁴⁰ | Benchtop photometer | Sample to answer | ~2 | Unknown* | No |

*Mode of operation unclear from available documentation and literature; reportable results provided between 2.0-12.0 mM

In various aspects, the test strip assembly may include the following:

I. A test strip assembly comprising:
a plasma separation membrane; and
a detection layer comprising a substrate treated with an optode coating solution,
wherein the test strip assembly is configured to selectively isolate a target ion in a sample and produce a photometric signal based upon the target ion concentration by exhaustively consuming the sample, the photometric signal being readable by an optoelectronic reader couplable to a handheld device.
   II. The test strip assembly of aspect I, wherein the optode coating solution comprises an ionophore selective to the target ion, an ion exchanger, and a proton-selective chromoionophore, and more specifically, wherein the target ion is potassium.
   III. The test strip assembly of aspect II, wherein the ionophore is selected from the group consisting of dibenzo-18-crown-6, cryptand-222, valinomycin, and combinations thereof.
   IV. The test strip assembly of any of aspects II or III, wherein the ion-exchanger is negatively charged.
   V. The test strip assembly of any of aspects II, III, or IV, wherein the ion-exchanger is sodium tetrakis [3,5-bis(trifluoromethyl)phenyl]borate].
   VI. The test strip assembly of any of aspects II, III, IV or V, wherein the proton-selective chromoionophore is selected from the group consisting of chromoionophore I, chromoionophore III, Ox B, and combinations thereof.
   VII. The test strip assembly of any of aspects II, III, IV, V or VI, wherein the proton-selective chromoionophore is chromoionophore.
   VIII. The test strip assembly of any of the above aspects, wherein the optode coating solution is an emulsion and further comprises a surfactant.
   IX. The test strip assembly of aspect VIII, wherein the optode coating solution does not comprise tetrahydrofuran.
   X. The test strip assembly of any of the above aspects, wherein the optode coating solution is coated onto polymer microspheres.
   XI. The test strip assembly of any of the above aspects, wherein the optode coating solution further comprises a lipophilic core material, an amphiphilic polymer, a carrier solvent, an excipient or a combination of these.
   XII. The test strip assembly of aspect XI, wherein the lipophilic core material comprises polystyrene, acrylic, poly(vinyl) chloride, or polyethylene glycol.
   XIII. The test strip assembly of any of the above aspects, wherein the detection layer further comprises a buffer.
   XIV. The test strip assembly of any of the above aspects, further comprising an application pad positioned relative to the separation membrane such that a sample applied to the application pad is conducted through the application pad and into contact with the separation membrane.
   XV. The test strip assembly of aspect XIV, further comprising a top film layer, a bottom film layer, one or more spacers, or a combination of these.
   XVI. The test strip assembly of aspect XV, wherein the top film layer defines an aperture, and the application pad is coupled to the top film layer below the aperture.
   XVII. The test strip assembly of aspect XVI, wherein the aperture and application pad define a reservoir having a sample volume capacity of from about 10 μ to about 50 μl.
   XVIII. The test strip assembly of aspect XVI, wherein the sidewalls of the aperture are configured to assist with sample flow.
   XIX. The test strip assembly of aspect XV, wherein the bottom film layer is optically clear.
   XX. The test strip assembly of aspect XV, wherein the bottom film layer is opaque and defines an aperture that allows optical access to the optode membrane substrate.

As mentioned previously, the devices, systems, and methods according to embodiments provide a quick, portable, minimally invasive, and cost efficient mechanism for measuring an electrolyte concentration, such as potassium, in a fluid sample such as blood, serum, or plasma for monitoring or diagnosing a condition in a patient or subject compared to those of the prior art. Unlike the devices and methods of the prior art, the devices, systems, and methods for determining the concentration of an electrolyte according to embodiments of the present invention utilize an efficient system without necessitating additional liquid handling steps by the end-user.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A test strip assembly comprising:
a plasma separation membrane; and
a detection layer comprising a substrate treated with an optode coating solution,
wherein the test strip assembly is configured to selectively isolate a target ion in a sample and produce a photometric signal based upon the target ion concentration by exhaustively consuming the sample, the photometric signal being readable by an optoelectronic reader couplable to a handheld device.

2. The test strip assembly of claim 1, wherein the optode coating solution comprises an ionophore selective to the target ion, an ion exchanger, and a proton-selective chromoionophore.

3. The test strip assembly of claim 2, wherein the target ion is potassium.

4. The test strip assembly of claim 3, wherein the ionophore is selected from the group consisting of dibenzo-18-crown-6, cryptand-222, valinomycin, and combinations thereof.

5. The test strip assembly of claim 3, wherein the ion-exchanger is negatively charged.

6. The test strip assembly of claim 5, wherein the ion-exchanger is sodium tetrakis [3,5-bis(trifluoromethyl)phenyl]borate].

7. The test strip assembly of claim 3, wherein the proton-selective chromoionophore is selected from the group consisting of chromoionophore I, chromoionophore III, Ox B, and combinations thereof.

8. The test strip assembly of claim 7, wherein the proton-selective chromoionophore is chromoionophore I.

9. The test strip assembly of claim 1, wherein the optode coating solution is an emulsion and further comprises a surfactant.

10. The test strip assembly of claim 9, wherein the optode coating solution does not comprise tetrahydrofuran.

11. The test strip assembly of claim 1, wherein the optode coating solution is coated onto polymer microspheres.

12. The test strip assembly of claim 1, wherein the optode coating solution further comprises a lipophilic core material, an amphiphilic polymer, a carrier solvent, an excipient or a combination of these.

13. The test strip assembly of claim 12, wherein the lipophilic core material comprises polystyrene, acrylic, poly(vinyl) chloride, or polyethylene glycol.

14. The test strip assembly of claim 1, wherein the detection layer further comprises a buffer.

15. The test strip assembly of claim 1, further comprising an application pad positioned relative to the separation membrane such that a sample applied to the application pad is conducted through the application pad and into contact with the separation membrane.

16. The test strip assembly of claim 15, further comprising a top film layer, a bottom film layer, one or more spacers, or a combination of these.

17. The test strip assembly of claim 16, wherein the top film layer defines an aperture, and the application pad is coupled to the top film layer below the aperture.

18. The test strip assembly of claim 17, wherein the aperture and application pad define a reservoir having a sample volume capacity of from about 10 μ to about 50 μl.

19. The test strip assembly of claim 17, wherein the sidewalls of the aperture are configured to assist with sample flow.

20. The test strip assembly of claim 16, wherein the bottom film layer is optically clear.

21. The test strip assembly of claim 16, wherein the bottom film layer is opaque and defines an aperture that allows optical access to the optode membrane substrate.

22. A method of manufacturing a test strip assembly comprising a plasma separation membrane and a detection layer comprising a substrate treated with an optode coating solution, the method comprising:
dispensing the optode coating solution onto beads, a substrate, or a combination of these to provide the detection layer, and
coupling the plasma separation membrane to the detection layer.

23. The method of claim 22, wherein the test strip assembly further comprises a top film layer having an aperture, an application pad positioned beneath top film layer, and spacers positioned on each side of the application pad, and the method further comprises coupling the top film layer, spacers and application pad to the detection layer and plasma separation membrane.

24. The method of claim 22, wherein the test strip assembly further comprises a bottom film layer, and the method further comprises coupling the bottom film layer to the detection layer on the side of the detection layer opposite the plasma separation membrane.

25. The method of claim 22, wherein the step of coupling comprises application of adhesive or tape.

26. The method of claim 22, wherein the test strip assembly is manufactured individually as a discrete test strip.

27. The method of claim 22, wherein a plurality of test strip assemblies are manufactured in roll form or large card format and thereafter separated into individual test strip assemblies.

28. An apparatus comprising:
a test strip assembly comprising a plasma separation membrane and a detection layer comprising a substrate treated with an optode coating solution; and
an optoelectronic reader for use with a mobile device having a jack plug receiving socket, said optoelectronic reader adapted for removably receiving the test strip,
wherein the test strip assembly is configured to selectively isolate a target ion in a sample and produce a photometric signal based upon the target ion concentration by exhaustively consuming the sample, the photometric signal being readable by the optoelectronic reader.

29. A method of measuring a target ion concentration using an optoelectronic reader operably coupled with a mobile device comprising:
receiving a bodily fluid sample on a test strip assembly, the test strip assembly comprising a plasma separation membrane and a detection layer comprising a substrate treated with an optode coating solution;
activating a light source to illuminate a reaction area of said test strip assembly in response to insertion of said test strip assembly into a test strip assembly receiving channel of the optoelectronic reader;
determining the target ion concentration of said bodily fluid sample based on the measured light reflected by the test strip assembly; and
transmitting a signal corresponding to said target ion concentration to said mobile device.

30. The method of claim 29, wherein the bodily fluid sample comprises blood, serum, plasma, urine, saliva, or a combination of these.

31. The method of claim 29, wherein the light source applies multiple wavelengths of illumination to the test strip assembly.

32. The method of claim 31, wherein the optode coating solution comprises a chromoionophore, and the light source supplies at least two wavelengths of illumination.

33. The method of claim 32, wherein the chromoionophore has a protonated and deprotonated form, and the absorption reflectance of the protonated form of the chromoionophore is measured at a first wavelength and the absorption reflectance of the deprotonated form of the chromoionophore is measured at a second wavelength.

34. A kit comprising an apparatus and instructions for using the apparatus, the apparatus comprising:
a test strip assembly comprising a plasma separation membrane and a detection layer comprising a substrate treated with an optode coating solution; and
an optoelectronic reader for use with a mobile device having a jack plug receiving socket, said optoelectronic reader adapted for removably receiving the test strip assembly,
wherein the test strip assembly is configured to selectively isolate a target ion in a sample and produce a photometric signal based upon the target ion concentration by exhaustively consuming the sample, the photometric signal being readable by the optoelectronic reader.

35. The kit of claim 34, wherein the instructions for using the apparatus include instructions for causing said mobile device to transmit a configuration profile to said optoelectronic reader.
